(19) United States
(12) Patent Application Publication
(10) Pub. No.: US 2024/0376209 A1
(43) Pub. Date: Nov. 14, 2024

(54) Method To Control High Molecular Weight Aggregates In An Antibody Composition

(71) Applicant: DR. REDDY’S LABORATORIES LIMITED, Hyderabad (IN)

(72) Inventors: Ram KUMAR M., Krishnagiri District (IN); Roja APUORVA T., Dharmapuri (IN); Ranjana SINGH, Chapra (IN); Kishore JAHAGIRDAR, Hyderabad (IN)

(21) Appl. No.: 18/291,909

(22) PCT Filed: Jul. 27, 2022
(86) PCT No.: PCT/IN2022/050677

§ 371 Date: Jan. 24, 2024

(30) Foreign Application Priority Data
Jul. 29, 2021 (IN) 202141034063

(51) Int. Cl. C07K 16/28; A61K 39/395; C07K 1/18

(52) U.S. Cl. CPC C07K 16/2839; A61K 39/39591; C07K 1/18

(57) ABSTRACT

The method disclosed in the current invention is used to purify an antibody from high molecular weight aggregates. The method discloses the use of anion exchange chromatography for the reduction of high molecular weight aggregates from the antibody composition, in particular, by contacting the antibody composition with the anion exchange resin at a specific pH and conductivity. The disclosed method eliminates the need for further chromatographic steps for the reduction of HMW aggregates.

### FIELD OF INVENTION

The present invention relates to protein purification methods. In particular, the invention relates to methods for purifying an antibody composition using ion exchange chromatography.

### BACKGROUND

Monoclonal antibodies (mAbs) are effective targeted therapeutic agents. The high specificity of the antibodies makes them ideal to reach their intended target and hence is useful to treat a wide variety of diseases.

The commercial production of recombinant human monoclonal antibody therapeutics demands robust processes, i.e., the purification scheme needs to reliably and predictably produce antibody composition intended for use in humans. A process should be designed to remove the product related contaminants such as high molecular weight (HMW) aggregates, product variants such as charged variants (acidic, deamidated/oxidized, basic), sequence variants and other species, as well as process related contaminants such as leached Protein-A, host cell protein, DNA, adventitious and endogenous viruses, endotoxin, extractable from resins and filters, process buffers and agents such as detergents that may have been employed for virus reduction. In designing a purification scheme and other conditions for each of the chromatographic steps, along with removal of contaminants, an important consideration is recovery from each step of the purification scheme and from the overall purification scheme. Hence, for a commercially viable process, the purification scheme needs to be designed to ensure adequate removal of contaminants from an antibody composition while maintaining the yield of the same.

Chromatographic techniques exploit the physical and chemical differences between the antibodies and the contaminant for the separation. Majority of purification schemes for mAbs involve a Protein-A based chromatography, which results in a high degree of purity and recovery in a single step. One or two additional chromatography steps are employed as polishing steps, generally selected from cation and anion exchange chromatography, although hydrophobic interaction chromatography, mixed mode chromatography or hydroxyapatite chromatography may be chosen as well.

Removal of aggregates, especially soluble aggregates, presents a challenge due to the physical and chemical similarity of the aggregates to the drug product itself, which is usually a monomer. Cation exchange chromatography (CEX), hydrophobic interaction chromatography (HIC), mixed-mode chromatography (MMC) or combinations thereof are generally used to control the HMW aggregates of antibodies. However, controlling of level of HMW aggregates becomes difficult if the process is devoid of chromatographic steps such as HIC and MMC and, specifically if the formation of HMW increases at any stage during the chromatographic or purification process.

Hence, there is a need for an improved process to control the HMW aggregates and product and process related impurities in the final drug substance of a therapeutic antibody composition.

### SUMMARY

The inventors of the current invention have surprisingly found that anion exchange chromatography in flow-through mode can be used to reduce the level of HMW aggregates in an antibody composition. Accordingly, the present invention discloses a method for the reduction of HMW aggregates in an antibody composition comprising the antibody and HMW aggregates by contacting the said antibody composition with an anion exchange support in the presence of a loading buffer and collecting the flow-through material from the anion exchange support, wherein the loading buffer has a pH of 7 and/or conductivity of 8 mS/cm or less.

The specific pH and/or the conductivity employed further effects a 10-fold reduction (1 log reduction) in the HCP content, further maintaining the recovery of the antibody to be about 95% or more.

The method disclosed as per the current invention is advantageous as it may not require further chromatographic steps such as HIC or MMC for the reduction of HMW aggregates.

### DETAILED DESCRIPTION OF THE INVENTION

### Definitions

The phrase “ion exchange material” refers to a solid phase which is negatively charged (i.e., a cation exchange resin) or positively charged (i.e., an anion exchange resin). The charge may be provided by attaching one or more charged ligands to the solid phase, e.g. by covalent linking. Alternatively, or in addition, the charge may be an inherent property of the solid phase (e.g. as is the case for silica, which has an overall negative charge).

The term “conductivity” refers to the ability of an aqueous solution to conduct an electric current between two electrodes. In solution, the current flows by ion transport. Therefore, with an increasing amount of ions present in the aqueous solution, the solution will have a higher conductivity. The unit of measurement for conductivity is mS/cm, and can be measured using a conductivity meter, e.g., by Orion. The conductivity of a solution may be altered by changing the concentration of ions therein. For example, the concentration of a buffering agent and/or concentration of a salt (e.g. NaCl or KCl) in the solution may be altered in order to achieve the desired conductivity.

A “contaminant” or “impurity”, as used interchangeably herein, is a material that is different from the desired polypeptide product. The contaminant may be a variant of the desired polypeptide (e.g. a deamidated variant or an aminoaspartate variant of the desired polypeptide) or another non-product related polypeptide, for e.g., host cell protein, host cell nucleic acid, endotoxin, etc. A contaminant can also be process related, for example—Protein-A-leachates.

“High molecular weight aggregates” as referred herein encompasses association of at least two molecules of a product of interest, e.g., antibody or any antigen-binding fragment thereof. The association of at least two molecules of a product of interest may arise by any means including, but not limited to, non-covalent interactions such as, e.g., charge-charge, hydrophobic and van der Waals interactions; and covalent interactions such as, e.g., disulfide interaction or non-reducible crosslinking. An aggregate can be a dimer, trimer, tetramer, or a multimer greater than a tetramer, etc.

The term “process or product related impurities” as used herein refer to the contaminants which may be derived from the manufacturing process, for example cell culture, downstream or cell substrates and may include host cell proteins, host cell DNA, nucleic acid, protein-A leachates etc., or may be molecular variants of the protein of interest, for example HMW aggregates, acidic variants, basic variants, low molecular weight variants etc., and may be formed during expression, manufacture or storage of the protein.

The term “about” as used herein, means an acceptable error range for the particular value as determined by one of ordinary skill in the art. For example, “about” can mean a range of up to 20%.

The “composition” to be purified herein comprises the protein of interest and one or more contaminants. The composition may be “partially purified” (i.e., having been subjected to one or more purification steps) or may be obtained directly from a host cell or organism producing the antibody (e.g., the composition may comprise harvested cell culture fluid).

The term “load” herein refers to the composition loaded onto the chromatography material, i.e., ion exchange support. Preferably, the chromatography material is equilibrated with an equilibration buffer prior to loading the composition which is to be purified.

The term “flow-through mode” as used herein refers to that process wherein the target protein is not bound to the chromatographic support but instead obtained in the unbound or “flow-through” fraction during loading or post-load wash of the chromatography support.

Aggregate concentration can be measured in a protein sample using Size Exclusion Chromatography (SEC), a well-known and widely accepted method in the art. Size exclusion chromatography uses a molecular sieving retention mechanism, based on differences in the hydrodynamic radii or differences in size of proteins. Large molecular weight aggregates cannot penetrate or only partially penetrate the pores of the stationary phase. Hence, the larger aggregates elute first and smaller molecules elute later, the order of elution being a function of the size.

### DETAILED DESCRIPTION OF THE EMBODIMENTS

The present invention discloses a method to purify an antibody composition comprising the antibody and contaminants, for example, high molecular weight aggregates, host cell proteins/nucleic acids, the method comprises the use of anion exchange chromatography.

In an embodiment, the method is used to reduce the level of high molecular weight aggregates in an antibody composition comprising an antibody and high molecular weight aggregates using anion exchange chromatography.

In another embodiment, the method is used to reduce the level of high molecular weight aggregates in an antibody composition comprising an antibody and high molecular weight aggregates using anion exchange chromatography, wherein the antibody composition is contacted with the anion exchange support in the presence of a loading buffer solution and the flow-through material is collected from the anion exchange support.

In another embodiment, the method disclosed in the invention is used to reduce the level of high molecular weight aggregates in an antibody composition, the method comprising steps of:
(a) contacting the antibody composition comprising the antibody and high molecular weight aggregates with the anion exchange support in the presence of a loading buffer solution,
(b) optionally washing the anion exchange support with a wash buffer solution,
(c) collecting the flow-through material from the anion exchange support,
wherein the loading buffer solution has a pH of about 7.

In yet another embodiment, the method disclosed in the invention is used to reduce the level of high molecular weight aggregates in an antibody composition, the method comprising steps of:
(a) contacting the antibody composition comprising the antibody and high molecular weight aggregates with the anion exchange support in the presence of a loading buffer solution,
(b) optionally washing the anion exchange support with a wash buffer solution,
(c) collecting the flow-through material from the anion exchange support,
wherein the loading buffer solution has a conductivity of 8 mS/cm or less.

In yet another embodiment, the method disclosed in the invention is used to reduce the level of high molecular weight aggregates in an antibody composition, the method comprising steps of:
(a) contacting the antibody composition comprising the antibody and high molecular weight aggregates with the anion exchange support in the presence of a loading buffer solution,
(b) optionally washing the anion exchange support with a wash buffer solution,
(c) collecting the flow-through material from the anion exchange support,
wherein the loading buffer solution has a conductivity of about 5 mS/cm.

In yet another embodiment, the method disclosed in the invention is used to reduce the level of high molecular weight aggregates in an antibody composition, the method comprising steps of:
(a) contacting the antibody composition comprising the antibody and high molecular weight aggregates with the anion exchange support in the presence of a loading buffer solution,
(b) optionally washing the anion exchange support with a wash buffer solution,
(c) collecting the flow-through material from the anion exchange support,
wherein the loading buffer solution has a pH of about 7 and a conductivity of 8 mS/cm or less.

In yet another embodiment, the method disclosed in the invention is used to reduce the level of high molecular weight aggregates in an antibody composition, the method comprising steps of:
(a) contacting the antibody composition comprising the antibody and high molecular weight aggregates with the anion exchange support in the presence of a loading buffer solution,
(b) optionally washing the anion exchange support with a wash buffer solution,
(c) collecting the flow-through material from the anion exchange support,
wherein the loading buffer solution has a pH of about 7 and a conductivity of about 5 mS/cm.

In yet another embodiment, the method disclosed in the invention is used to reduce the level of process and product related impurities, including high molecular weight aggregates, in an antibody composition, the method comprising steps of:
(a) contacting the antibody composition comprising the antibody and high molecular weight aggregates with the anion exchange support in the presence of a loading buffer solution,
(b) optionally washing the anion exchange support with a wash buffer solution,
(c) collecting the flow-through material from the anion exchange support,
wherein the loading buffer solution has a pH of about 7 and a conductivity of 8 mS/cm or less.

In the abovementioned embodiment, the product and process-related impurities are selected from—host cell DNA, host cell proteins, protein-A leachates, endotoxins, and low molecular weight species.

In any of the above mentioned embodiments, the high molecular weight aggregates are reduced by at least 50% in the flow-through material collected from the anion exchange support as compared to the level of high molecular weight aggregates in the antibody composition loaded onto the anion exchange support.

In any of the above mentioned embodiments, the method is also used to reduce the level of other process-related impurities, including, but not limited to, endotoxins, protein-A leachates, host cell proteins, host cell DNA, etc.

In any of the above mentioned embodiments, the method is used to reduce the level of HCPs in the antibody composition by about 10-folds.

In any of the above mentioned embodiments, the amount of host cell DNA in the flow-through material is about 0.4 pg/mg.

In any of the above mentioned embodiments, the recovery of the antibody in the flow-through is not less than 90%.

In any of the above mentioned embodiments, the recovery of the antibody in the flow-through is about 95% or more.

In any of the above mentioned embodiments, the loading buffer solution is phosphate buffer.

In any of the above mentioned embodiments, the pH and conductivity of the wash buffer solution is same as that of the loading buffer solution.

In any of the above mentioned embodiments, the pH and conductivity of the wash buffer solution is different than that of the loading buffer solution.

In any of the above mentioned embodiments, AEX is the last chromatographic step in the purification process.

In any of the above mentioned embodiments, the AEX step is immediately preceded by tangential flow filtration step.

In any of the above mentioned embodiments, the antibody is an anti-α4β7 antibody or antigen binding fragment thereof.

In any of the above mentioned embodiments, the antibody is vedolizumab.

The invention is more fully understood by reference to the following examples. These examples should not, however, be construed as limiting the scope of the invention.

### EXAMPLES

### Example 1

A therapeutic monoclonal antibody which binds to human α4ß7 integrin was cloned and expressed in a Chinese Hamster Ovary cell line and the cell culture broth containing the expressed antibody was harvested, clarified and subjected to protein-A affinity chromatography. The process was carried out initially at a 50-liter scale and then it was scaled up to 1000-liters. The eluate from protein-A affinity chromatography was subjected to low-pH incubation and depth filtration, and further polishing steps, including cation exchange chromatography (CEX). The eluate obtained from CEX was subjected to tangential flow filtration (TFF) and the retentate of TFF was used as the load for anion exchange chromatography (AEX). At this stage, HMW aggregate level was determined using analytical size exclusion chromatography and was found to be significantly increased as compared to the level of HMW aggregates in the previous step, i.e., CEX eluate (see Table 3). AEX was then carried out in flow-through mode and the specific pH and conductivity of the loading buffer was used to reduce the level of HMW aggregates. Details of AEX chromatography are given in Tables 1 and 2. It is to be noted that samples Vmab-1, Vmab-2 and Vmab-3 were obtained from the 50-liter scale, whereas samples Vmab-4 and Vmab-5 were obtained from the 1000-liter scale.

TABLE 1
Chromatography conditions used in AEX

| Resin | Q Sepharose FF |
|---|---|
| Bed Height (cm) | 18-22 |
| Residence Time (min) | 6.0 |
| (loading and Post-load wash) | |
| Load Factor (g/L) | 150 |

TABLE 2
Details of buffer used in AEX

| | | | Conductivity |
|---|---|---|---|
| Stage | Buffer | pH | (mS/cm) |
| Pre- | 200 mM phosphate buffer, | 6.30 | 120 ± 5 |
| equilibration | 1.5M NaCl | | |
| Equilibration | 35 mM phosphate buffer | 7.0 | 4 ± 1 |
| Post load wash | 35 mM phosphate buffer | 7.0 | 4 ± 1 |
| Regeneration | 0.5M NaOH | NA | NA |

Table 3 shows the HMW aggregate data at the TFF input and output stages.

TABLE 3
HMW level at TFF stage

| | HMW % | HMW % | % |
|---|---|---|---|
| Batch | (TFF input) | (TFF output) | increase |
| Vmab-1 | 0.3 | 0.6 | 100% |
| Vmab-2 | 0.3 | 0.6 | 100% |
| Vmab-3 | 0.3 | 0.6 | 100% |
| Vmab-4_cycle 1 | 0.3 | 0.7 | 133% |
| Vmab-5_cycle 2 | 0.3 | 0.7 | 133% |
| Vmab-5_cycle 1 | 0.2 | 0.4 | 100% |
| Vmab-5_cycle 2 | 0.2 | 0.4 | 100% |

The flow-through material obtained from AEX was subjected to analytical SEC to determine the level of HMW aggregate. Table 3 summarizes the HMW aggregate level at the time of loading onto AEX and in the flow-through obtained from AEX.

TABLE 4
HMW aggregate level in AEX load and AEX flow-through stages

| | HMW % | HMW % | % |
|---|---|---|---|
| Batch | (load) | (flow-though) | Reduction |
| Vmab-1 | 0.7 | 0.3 | 57% |
| Vmab-2 | 0.7 | 0.3 | 57% |
| Vmab-3 | 0.6 | 0.3 | 50% |
| Vmab-4_cycle 1 | 0.7 | 0.3 | 57% |
| Vmab-4_cycle 2 | 0.7 | 0.3 | 57% |
| Vmab-5_cycle 1 | 0.4 | 0.1 | 75% |
| Vmab-5_cycle 2 | 0.4 | 0.2 | 50% |

Similarly, the levels of HCP and protein-A leachates were determined at the AEX load and flow-through stages and are represented in Table 4 along with HCD content in AEX flow-through.

TABLE 5
HCP and PAL levels at AEX load and AEX FT stages

| | HCP (ng/mg) | | PAL (ng/mg) | | HCD (pg/mg) |
|---|---|---|---|---|---|
| Batch | AEX | AEX | AEX | AEX | AEX |
| number | load | FT | load | FT | FT |
| Vmab-1 | 4.50 | 0.40 | 3.10 | 0.10 | 0.50 |
| Vmab-2 | 5.51 | 0.40 | 4.30 | 0.10 | 0.20 |
| Vmab-3 | 4.20 | 0.40 | 3.00 | 0.10 | 0.10 |
| Vmab-4_cycle 1 | 5.0 | <1.1 | <1.15 | <0.11 | <0.85 |
| Vmab-4_cycle 2 | 4.9 | <1.2 | <1.18 | <0.13 | <0.97 |
| Vmab-5_cycle 1 | 4.8 | <1.1 | <1.04 | <0.11 | <0.88 |
| Vmab-5_cycle 2 | 4.8 | <1.2 | <1.14 | <0.12 | <0.96 |

It is evident from Tables 4 and 5 that the disclosed method is able to significantly reduce the levels of HMW aggregate in addition to the removal of HCP and protein-A leachates.

1. 1. A method for reduction of high molecular weight aggregates from an anti-α4β7 antibody composition, comprising the steps of
(a) contacting the antibody composition comprising an anti-α4β7 antibody and high molecular weight aggregates thereof with an anion exchange support in the presence of a loading buffer solution,
(b) optionally washing the anion exchange support with a wash buffer solution, and
(c) collecting the flow-through material from the anion exchange support,
wherein the loading buffer solution has a pH of about 7 and a conductivity of 8 mS/cm or less.

2. The method as claimed in claim 1, wherein the conductivity of the loading buffer solution is 5 mS/cm.

3. The method as claimed in claim 1, wherein the high molecular weight aggregates are reduced by up to 75%, or up to 65%, or up to 55%, or at least by 50% in the flow-through material collected from the anion exchange support as compared to the level of high molecular weight aggregates in the antibody composition loaded onto the anion exchange support.

4. The method as claimed in claim 1, wherein the amount of antibody recovered in the flow-through material is about 95% or more.

5. The method as claimed in claim 1, wherein the loading buffer solution comprises phosphate.

6. The method as claimed in claim 1, wherein the anti-α4β7 antibody is vedolizumab.

7. The method as claimed in claim 1, wherein the pH and conductivity of the wash buffer solution and the loading buffer solution is same.

8. The method as claimed in claim 1, wherein the antibody composition is subjected to a tangential flow filtration step prior to contacting with the anion exchange support.
